(19) United States
(12) Patent Application Publication
(10) Pub. No.: US 2023/0326546 A1
(43) Pub. Date: Oct. 12, 2023

(54) INTERACTIVE AMINO ACID SEQUENCE GUIDE

(71) Applicants: PIERCE BIOTECHNOLOGY, INC., Rockford, IL (US); LIFE TECHNOLOGIES CORPORATION, Carlsbad, GA (US)

(72) Inventors: Brian Johnson, Rockton, IL (US); Rachel Samuelson, Rockford, IL (US); Matthew Roxo, Escondido, CA (US)

(73) Assignees: PIERCE BIOTECHNOLOGY, INC., Rockford, IL (US); LIFE TECHNOLOGIES CORPORATION, Carlsbad, CA (US)

(21) Appl. No.: 18/118,630

(22) Filed: Mar. 7, 2023

Related U.S. Application Data
(60) Provisional application No. 63/317,950, filed on Mar. 8, 2022.

(51) Int. Cl. G16B 15/30; G06F 3/04847

(52) U.S. Cl. CPC G16B 15/30; G06F 3/04847

(57) ABSTRACT

Methods and systems for generating a computer-implemented interactive graphical user interface for facilitating finding appropriate antibody products utilizing visual evaluation of sequence information are disclosed. An antibody product listing within the graphical user interface is generated. The antibody product listing is responsive to a user interaction. The antibody product listing includes at least one antibody product including corresponding sequence information and product specifications. A sequence infographic is generated based on the corresponding sequence information, wherein the sequence infographic includes an amino acid sequence area of interest indicator. The sequence infographic is displayed aligned relative to a plurality of sequence infographics.

### TECHNICAL FIELD

This disclosure relates generally to online or other electronic systems for accessing antibody product information and identifying and obtaining relevant antibody products.

### BACKGROUND

Antibody products are cataloged and provided to help researchers in many fields including, for example, cellular analysis, genomics, proteomics, drug discovery, biodefense, environmental diagnostics, epigenetics, and stem cell research. Existing online or other electronic catalogs readily provide product availability and price information. However, other important product information is not provided in a format that is sufficiently easy to digest and compare across large numbers of antibody products.

### SUMMARY

Scientists and researchers, as consumers of antibody products, generally need to analyze information about a particular product's characteristics beyond availability and price. Scientists may also be interested in determining the availability of all of the products that are related to their area of research, for example, all of the products that might be used to study a particular amino acid area of interest within a target protein.

However, using presently available systems, finding the correct antibody product to use in a particular experiment is time consuming and cumbersome. Information regarding the antigen or immunogen used to create the antibody product is critical for identifying the best antibody products for a particular purpose. Current systems, however, have inadequate tools for providing this information. Such information is typically buried in product descriptions, and scientists and researchers must manually parse through multiple product descriptions, one-by-one, to determine antigen or immunogen information. The time-consuming aspect of these searches is compounded by having to review text-based information from a detailed description of each antibody product and not being able to easily compare this information between products.

Accordingly, there is a need for an intelligent, customized, and user-friendly visual representation of large amounts of amino acid sequence data related to antibody products, that is easy to access, interpret and compare by scientists and researchers.

Embodiments of the present invention relate to an interactive user interface for antibody products. The interface enables visual evaluation of immunogen or antigen information. Some of the embodiments provide a specific, structured graphical user interface paired with a prescribed functionality directly related to the graphical user interface's structure for determining the correct antibody product. Some embodiments provide specific structures such as infographics that allow a user to avoid the burdensome task of navigating through each product and reviewing each product description in separate windows to identify and compare sequence information.

Certain embodiments may provide one or more of the following technical advantage(s). Advantages and efficiencies are obtained from presenting the detailed information in a compact visual format. Disclosed embodiments provide dramatic improvements in reviewing, analyzing, evaluating, and comparing immunogen or antigen information corresponding to antibody products by using user friendly infographics conveying amino acid sequence information for each product that is otherwise difficult to digest, analyze or visualize, and especially compare between antibody products. The infographics are provided in an organized and compact format, conveying information in a glance that was previously buried and disorganized. Visual interactive elements of user interface embodiments further facilitate efficiently searching for and identifying relevant antibody products. In this manner, complexities of immunogen or antigen information are transformed into a simple to understand visual format, providing a highly intuitive interface, one in which advanced features, for instanceamino acid sequence information, are easily discernable.

Various computer-implemented systems, methods, and articles of manufacture for an interactive user interface for generating a computer-implemented interactive graphical user interface for facilitating finding appropriate antibody products utilizing visual evaluation of immunogen or antigen information, are described herein that can improve determining a correct antibody product for a particular experiment with respect to the various challenges mentioned above.

Various objects, features, aspects, and advantages of the inventive subject matter will become more apparent from the following specification, along with the accompanying drawings in which like numerals represent like components.

### BRIEF DESCRIPTION OF THE DRAWINGS

FIG. 1 is a flowchart illustrating a method for generating a computer-implemented interactive graphical user interface for facilitating finding appropriate antibody products utilizing visual evaluation of immunogen or antigen information according to various embodiments.

FIG. 2 is a flowchart illustrating a method for generating a computer-implemented interactive graphical user interface for facilitating finding appropriate antibody products utilizing visual evaluation of immunogen or antigen information according to various embodiments.

FIG. 3 is a block diagram illustrating interactive interface components for generating a computer-implemented interactive graphical user interface for facilitating finding appropriate antibody products utilizing visual evaluation of immunogen or antigen information according to various embodiments.

FIG. 4 is a block diagram illustrating an interactive user interface for generating a computer-implemented interactive graphical user interface for facilitating finding appropriate antibody products utilizing visual evaluation of immunogen or antigen information according to various embodiments.

FIG. 5 is a block diagram illustrating an interactive user interface for generating a computer-implemented interactive graphical user interface for facilitating finding appropriate antibody products utilizing visual evaluation of immunogen or antigen information according to various embodiments.

FIG. 6 is a block diagram illustrating an interactive user interface for generating a computer-implemented interactive graphical user interface for facilitating finding appropriate antibody products utilizing visual evaluation of immunogen or antigen information according to various embodiments.

FIGS. 7A-7C are block diagrams illustrating interactive amino acid sequence sliders for generating a computer-implemented interactive graphical user interface for facilitating finding appropriate antibody products utilizing visual evaluation of immunogen or antigen information according to various embodiments.

FIGS. 8A-8D are block diagrams illustrating antibody product infographics for generating a computer-implemented interactive graphical user interface for facilitating finding appropriate antibody products utilizing visual evaluation of immunogen or antigen information according to various embodiments.

FIG. 9 is a block diagram illustrating an interactive user interface for generating a computer-implemented interactive graphical user interface for facilitating finding appropriate antibody products utilizing visual evaluation of immunogen or antigen information according to various embodiments.

FIG. 10 is a block diagram illustrating an interactive user interface for generating a computer-implemented interactive graphical user interface for facilitating finding appropriate antibody products utilizing visual evaluation of immunogen or antigen information according to various embodiments.

FIG. 11 is a block diagram illustrating an interactive user interface for generating a computer-implemented interactive graphical user interface for facilitating finding appropriate antibody products utilizing visual evaluation of immunogen or antigen information according to various embodiments.

FIG. 12 is a block diagram illustrating a three-dimensional (3D) interactive filter for generating a computer-implemented interactive graphical user interface for facilitating finding appropriate antibody products utilizing visual evaluation of immunogen or antigen information according to various embodiments.

FIG. 13 is a block diagram illustrating a 3D infographic for generating a computer-implemented interactive graphical user interface for facilitating finding appropriate antibody products utilizing visual evaluation of immunogen or antigen information according to various embodiments.

FIG. 14 illustrates an exemplary computer system configurable by a computer program product for an interactive user interface for generating a computer-implemented interactive graphical user interface for facilitating finding appropriate antibody products utilizing visual evaluation of immunogen or antigen information to carry out embodiments of the present invention.

While the invention is described with reference to the above drawings, the drawings are intended to be illustrative, and other embodiments are consistent with the spirit, and within the scope, of the invention.

### DETAILED DESCRIPTION

To provide a more thorough understanding of the present invention, the following description sets forth numerous specific details, such as specific configurations, parameters, examples, and the like. It should be recognized, however, that such description is not intended as a limitation on the scope of the present invention but is intended to provide a better description of the exemplary embodiments.

According to various embodiments, the present disclosure may be directed to devices, instruments, systems, and methods for an interactive user interface for generating a computer-implemented interactive graphical user interface for facilitating finding appropriate antibody products utilizing visual evaluation of immunogen or antigen information.

One should appreciate that the disclosed techniques provide many advantageous technical effects including an interactive user interface for generating a computer-implemented interactive graphical user interface for facilitating finding appropriate antibody products utilizing visual evaluation of immunogen or antigen information.

One should appreciate that the disclosed techniques provide many advantageous technical effects including interactive computer interface methods for visually reviewing, identifying and comparing antibody products. The disclosed techniques have been designed to support reviewing, identifying and comparing accuracy on a scale and speed that cannot be achieved using manual human effort.

It should also be appreciated that the following specification is not intended as an extensive overview, and as such, concepts may be simplified in the interests of clarity and brevity.

Search is critical for researchers to find the correct antibody to use in a given experiment. Many factors figure into the decision of which antibody antibodies to choose. Some factors are easily summarized in a table and lend themselves to standard filtering and sorting. Information related to antigens or immunogens does not lend itself to this format. The antigen/immunogen originally used to create an antibody can be a critical factor in choosing the right antibody. This information is typically buried in the details listed on individual product pages and not easily compiled, reviewed and compared.

The disclosure provides a graphical interface that enables a user to quickly view, filter, sort, or compare a list of antibodies according to the antigen/immunogen. The disclosure provides user friendly access to immunogen information in a graphical interface that allows for quick filtering, sorting, and comparison among antibody products.

The disclosure provides an antigen/immunogen search utility in a web-based antibody search interface that converts text/numerical information for the sequence of a given target protein, and the antigens/immunogens used to generate antibodies to that target protein, into a graphical format that is presented in a visual interface.

The disclosure enables users to quickly view and compare choices across all available antibody options on a single page and in a filterable format that also includes the ability to filter results by position within the full-length target protein.

Interface elements are visually distinguished through shape, color, size, length, position, location, grouping, or behavior, or by aligning or juxtaposing associated elements or signifying indicators in order to help a user identify a particular antibody product based on an amino acid area of interest or fragment of interest within the full-length amino acid sequence of the target protein. This differentiation may be done by the content provider or by the user.

Interface elements are used to provide visual information related to an amino acid sequence related to a target protein. The amino acid sequence has a starting amino acid and ending amino acid. Proteins are composed of a linear chain of amino acids linked to one another by a bond. At the start of the protein is the N-terminus and at the end of the protein is the C-terminus. The N-terminus is the free end of the chain terminating in a carboxyl group, and the C-terminus is the free end of carboxylic acids, the two acid groups being linked together through the length of the protein chain. The amino acid sequence is written or displayed from left to right, N-terminus to C-terminus.

While the term generating is used to describe presenting the infographics or interface elements, the infographics may be pre-generated, stored, retrieved and displayed on demand. Further, the interface elements or infographics may be images, videos, digital layers, virtual representations, haptics, or a combination thereof.

The graphical user interface includes antibody product representations. The user is searching for one or more antibody products based on amino acid area or fragment of interest related to each product. Each antibody has been made against a protein or a sequence that corresponds to part of a protein. The immunogen has been injected into an animal to raise an antibody response. The resulting antibodies bind to a specific antigen, or peptide sequence, contained within the target protein.

In this disclosure, antigen or immunogen may refer to an amino acid sequence relevant to producing a particular antibody product. In this disclosure, protein and target may be used interchangeably and may also refer to a full-length protein, which was the target to which the antibody product was made.

The graphical user interface facilitates identification of the antigen or immunogen corresponding to the antibody product and provides a quick and easy way to visually sort through the information related to the antigens or immunogens. The amino acid sequence infographic provides an easy way to determine what part of the protein the corresponding antibody product might recognize and react against.

When epitope information is available for a particular antibody product, the infographic may include a visual indication for the epitope information. The epitope is usually smaller and more specific than the amino acid sequence representing the antigen or immunogen. The epitope is the specific binding site of the antibody. That is, within the target protein the epitope is a small area that the antibody recognizes and binds to. For example, a full protein may be used as an immunogen, but the antibody, especially in the case of a monoclonal antibody might only recognize a specific portion of the protein target, called the epitope. In the case of polyclonal antibodies, there will not be a specific epitope and the antibody may bind to multiple regions of the protein. In some embodiments, the epitope is not known and only an approximate antigen sequence is known.

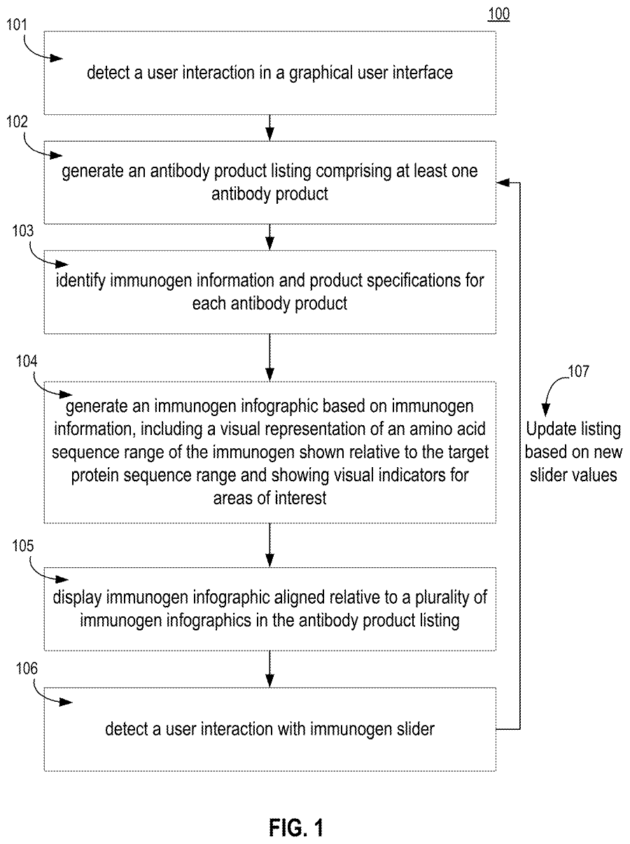

FIG. 1 is a flowchart illustrating a method 100 for generating a computer-implemented interactive graphical user interface for facilitating finding appropriate antibody products utilizing visual evaluation of immunogen or antigen information.

In one embodiment, method 100 for generating a computer-implemented interactive graphical user interface for facilitating finding appropriate antibody products utilizing visual evaluation of immunogen or antigen information begins with step 101.

At step 101, the method detects a user interaction in a graphical user interface. User interaction includes a user event with respect to the interactive graphical interface.

At step 102, the method generates an antibody product listing comprising at least one antibody product. The antibody product listing includes antibody products and corresponding illustrations or infographics.

At step 103, the method identifies immunogen information and product specifications for each antibody product. Immunogen information includes amino acid sequence information.

At step 104, the method generates an immunogen infographic based on immunogen information, including a visual representation of an amino acid sequence range or fragment of the immunogen shown relative to the target protein sequence range or fragment and showing visual indicators for areas of interest. Areas of interest include an amino acid sequence range or fragment which includes amino acids that the antibody product was derived from.

At step 105, the method displays immunogen infographics aligned relative to a plurality of immunogen infographics in the antibody product listing. This visual alignment provides a visual reference point for the amino acid areas of interest relative to the reference protein sequence as well as relative to other antibody product immunogens/antigens. In some embodiments, visual alignment includes alignment in multiple orientations or dimensions including for example vertical alignment, horizontal alignment, forward alignment and backward alignment, among others.

At step 106, the method detects a user interaction with immunogen slider. A user interaction includes any user-initiated event related to navigating an interactive interface. The interaction includes moving the slider, or interacting with a constraint indicator, which corresponds to bounding values or constraining values along the reference target protein which is configured as a filter element in the interface. The amino acid value indicated by the slider indicator will be used to determine which products to filter in the product listing.

At step 107, the method updates the product listing based on new slider values. For example, if the new slider values indicate the area or fragment of interest is between amino acids 20 and 100, then the product listing will be updated to include products where immunogens/antigen amino acid sequence values are between 20 and 100 and exclude products with amino acid values lower than 20, and higher than 100.

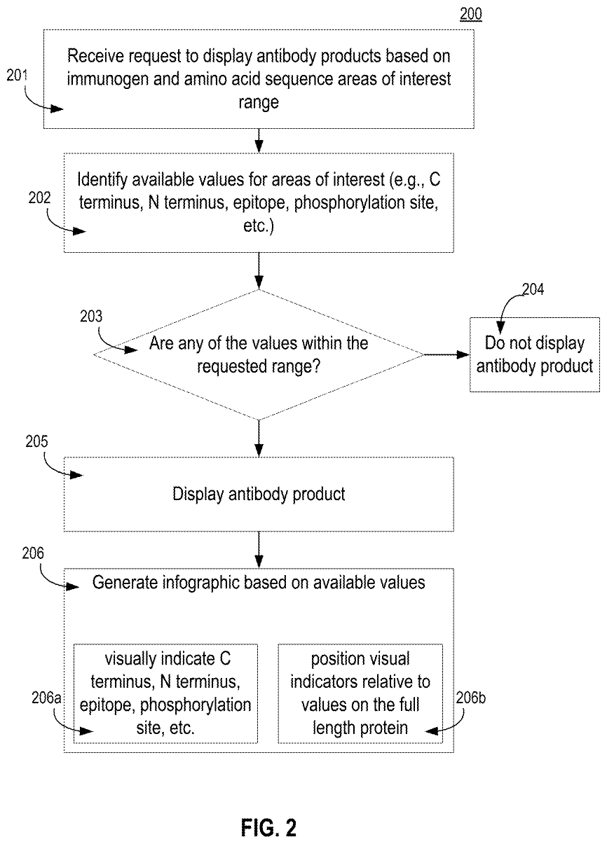

FIG. 2 is a flowchart illustrating a method 200 for generating a computer-implemented interactive graphical user interface for facilitating finding appropriate antibody products utilizing visual evaluation of immunogen or antigen information. In one embodiment, method 200 begins with step 201, during which the computer-implemented interactive graphical user interface receives a request to display antibody products based on immunogen and amino acid sequence areas of interest range. In some embodiments, the request may be initiated by a user interaction. In some embodiments, the request may be triggered by an automated computerized process.

At step 202, the method identifies available values for areas of interest, such as, but not limited to starting amino acid, ending amino acid, amino acid range of interest, estimated amino acid area or fragment of interest, epitope, and phosphorylation site.

At step 203, the method queries each product in the product listing to determine whether there are any products where the values of the antigen/immunogen amino acid values are within the requested range.

At step 204, if the product is outside the requested range, then the method will not display the antibody product. In other words, the antibody product will be filtered out.

At step 205, if the product is inside the requested range, then the method will display the antibody product. In other words, the antibody product will not be filtered out and will be included in the product listing.

At step 206, the method will generate an infographic based on available values for each product included in the product listing. In some embodiments, the infographic may be pre-generated, stored and retrieved on demand. The infographic will include all available values related to the areas of interest such as but not limited to starting amino acid, ending amino acid, amino acid range of interest, estimated amino acid area of interest, epitope, and phosphorylation siteln some embodiments, a toggle or another interface element may be used to control which values in the areas of interest should be generated as part of the infographic. In some embodiments, a toggle or another interface element may be used to identify whether to display products that include particular values related to particular areas of interest. For example, there may be a toggle to only display products that have epitope information. As another example, there may be a toggle to display or not display epitope information, based on availability of the amino acid value of the epitope.

At step 206a, the method visually indicates C-terminus, N-terminus, epitope, phosphorylation site, etc. For example, areas of interest are visually indicated to provide information about particular available values related to the immunogen/antigen. Other examples of elements that may be visually indicated when available include starting amino acid, ending amino acid, amino acid range of interest, estimated amino acid area of interest, epitope, and phosphorylation site, among others.

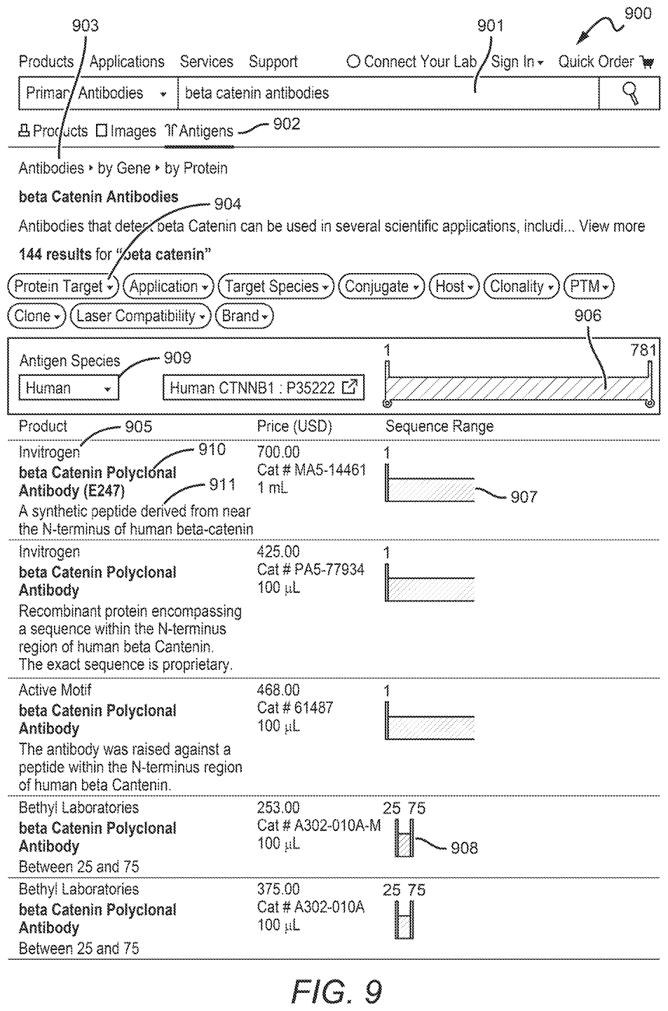

At step 206b, the method positions visual indicators relative to values on the full-length protein. For example, all of the infographics may be presented one under the other and under the full-length target protein and visually positioned based on their respective amino acid values. Thus, as shown in FIGS. 9, 10, and 11, for example, the starting and ending amino acids shown in each infographic will be visually positioned relative to those shown for the other infographics and relative to a visual indicator of the full-length target protein sequence and/or a user-selected sequence range within the full-length target protein sequence.

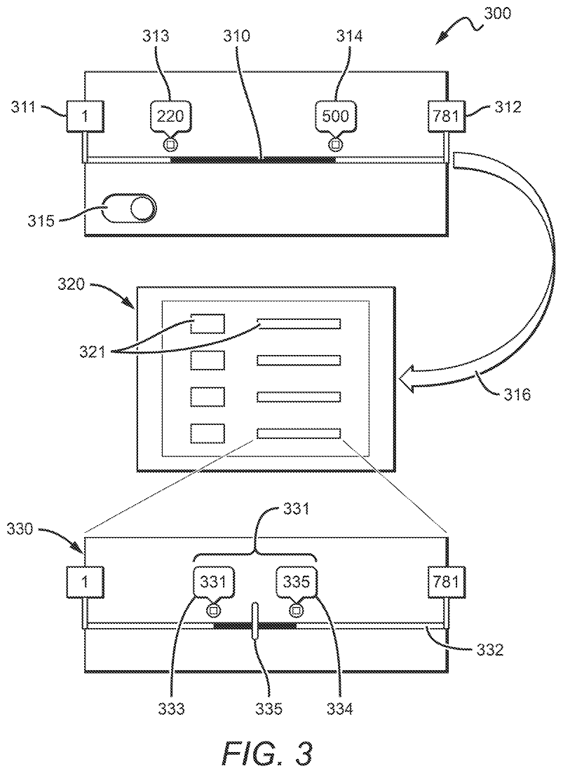

FIG. 3 is a block diagram illustrating interactive interface components for generating a computer-implemented interactive graphical user interface for facilitating finding appropriate antibody products utilizing visual evaluation of immunogen or antigen information according to various embodiments. In one embodiment, interactive interface components 300 include interactive sequence slider 310, antibody product listing 320 and amino acid sequence infographic 330. Interactive sequence slider 310 includes N-terminus 311, C-terminus 312, starting amino acid 313, ending amino acid 314 and toggle 315.

In some embodiments, toggle 315 controls whether additional information on the infographic is displayed. As one example, additional information includes epitope information and toggle 315 controls whether to display additional information via indicator 335. In some embodiments, the additional information may be overlayed on top of, or partially overlapping amino acid sequence infographic 330. In some embodiments, overlaying includes blending, superimposing, layering or merging multiple images or information indicators.

User interaction with interactive sequence slider 310 results in updating the listing 316, for example, including updating antibody product listing 320 and/or updating protein sequence information and product specifications 321, which is included in antibody product listing 320.

Amino acid sequence infographic 330 includes starting amino acid 333, ending amino acid 334, indicator 335, area of interest 331, and full amino acid sequence range 332. In some embodiments, indicator 335 includes epitope information.

In some embodiments, visual representation of amino acid sequence information related to or included as part of infographic 330 may be overlayed, blended, superimposed, or merged with the visual representation of the infographic. Amino acid sequence information includes starting amino acid 333, ending amino acid 334, indicator 335, and full amino acid sequence range 332, area of interest 331, bounding or constraint elements, amino acid range of interest, estimated amino acid area of interest, epitope, phosphorylation site, etc.

In some embodiments, data overlays may be used to include more information in a visual format. Additional information related to areas of interest may be overlaid on the interface element representing the target protein, including an image, a video, or another type of interface element. For example, data overlays may include epitope information for the antibody product, or multiple product information.

Overlay antigen or immunogen data may include, for example, bounding or constraining elements such as starting and/or ending amino acid, epitope, binding area or fragment of interest or bounding or constraining interval, as an overlay layer on a base layer such as a full length protein. By use of the overlay, the user can quickly see which areas of the protein the antibody was made against. The coordinates of the overlay correspond to and align on the amino acids of the full-length protein.

In some embodiments, interactive interface components include a generated multi-dimensional protein model including a visual representation of at least one level of protein structure. In some embodiments, the protein structure includes one of a primary protein structure, a secondary protein structure, a tertiary protein structure and a quaternary protein structure. The interactive interface may position the amino acid sequence area of interest indicator on the at least one level of protein structure.

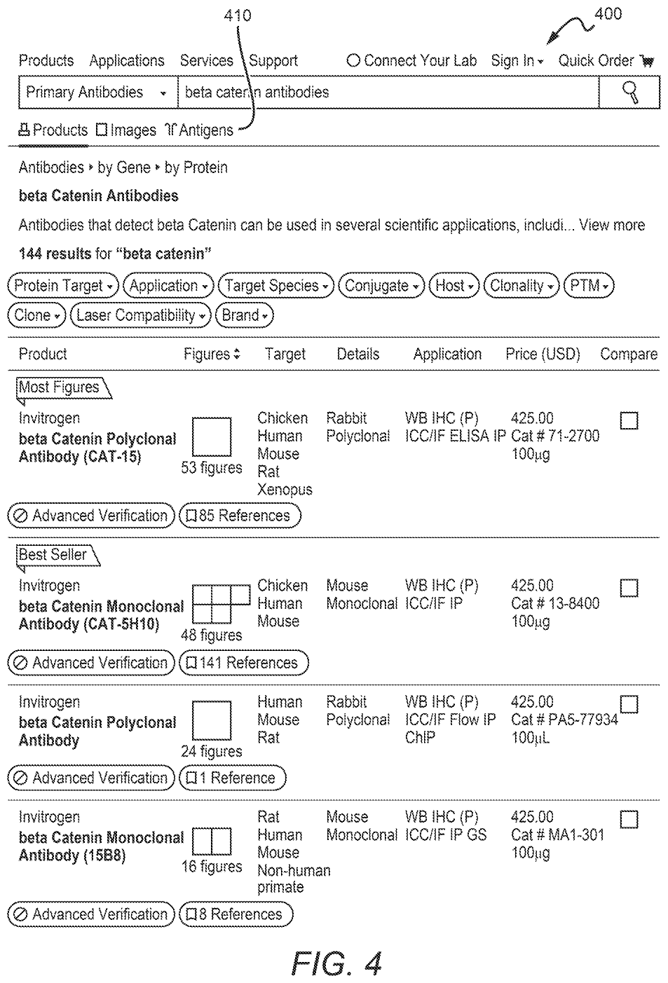

FIG. 4 is a block diagram illustrating an interactive user interface for generating a computer-implemented interactive graphical user interface for facilitating finding appropriate antibody products utilizing visual evaluation of immunogen or antigen information according to various embodiments. In one embodiment, products page 400 is configured to display products information for the beta Catenin target family.

Navigation options 410 include options for a products page, images page, and antigens page. Products is underlined in navigation options 410, which indicates that the user is currently on the products page. In some embodiments, a user event, for example, hovering a pointer over a particular option or element in navigation options 410 may draw attention or highlight to the option by underlining the option, including for example, using color or another visually distinguishing characteristic to highlight the option. In some embodiments, when the Antigens option is clicked or selected, the user is taken to the antigen product listing page.

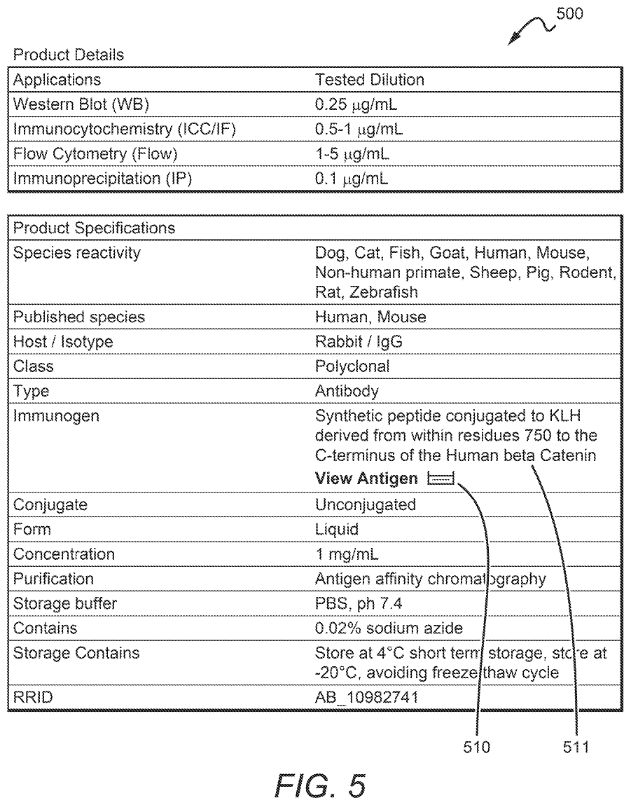

FIG. 5 is a block diagram illustrating an interactive user interface for generating a computer-implemented interactive graphical user interface for facilitating finding appropriate antibody products utilizing visual evaluation of immunogen or antigen information according to various embodiments. In this example, interactive interface 500 is a product page for a beta Catenin polyclonal antibody, including antigen description 511. From this page, a user may use antigen link 510 to navigate to an expanded information view for the antibody product, including for example, an amino acid infographic view including the amino acid sequence range for the antigen or immunogen, against the full-length protein.

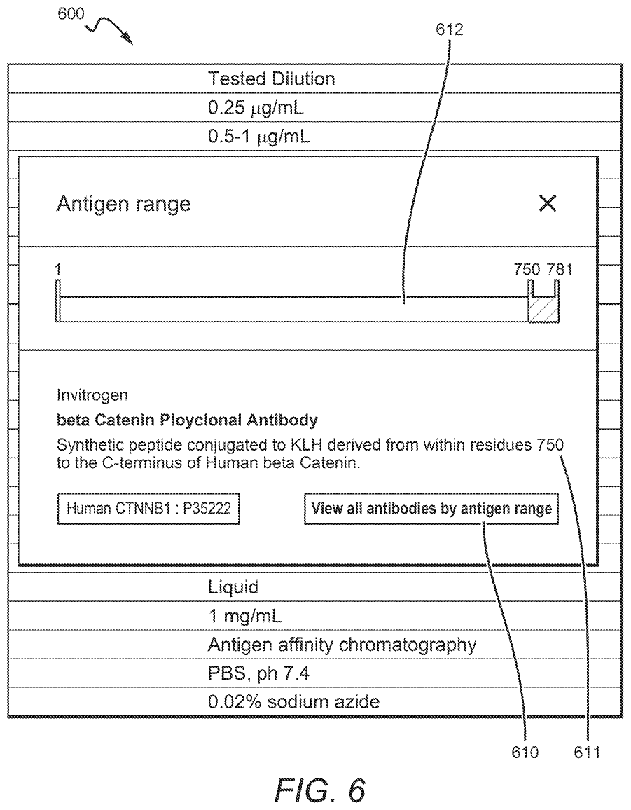

FIG. 6 is a block diagram illustrating an interactive user interface for generating a computer-implemented interactive graphical user interface for facilitating finding appropriate antibody products utilizing visual evaluation of immunogen or antigen information according to various embodiments. In one embodiment, interactive interface 600 is a product page for a beta Catenin polyclonal antibody, including description 611 and amino acid infographic 612.

In some embodiments, amino acid infographic 612 is displayed in response to a user interaction event, including, for example, clicking on link 510 which causes dynamically expanding the viewing area to include additional information associated with the antigen or immunogen corresponding to the antibody product.

Amino acid infographic 612 includes immunogen or antigen information and provides a visual representation of an immunogen range against the full-length protein, for example amino acid range 750-781. From this page, a user may navigate to listing link 610, by clicking “View all antibodies by antigen range.” After clicking listing link 610, a user is taken to a page including antibody product listings for beta Catenin.

In some embodiments, a user can view the immunogen or antigen range against the full-length protein in amino acid infographic 612. In this example, the full-length protein range of the target protein is from amino acid 1 to amino acid 781 and the immunogen or antigen is between amino acid 750 and amino acid 781. The information about how the antibody was derived is also described in description 611. In some embodiments, the free text description information is used to parse antigen or immunogen amino acid related information, such as starting amino acid and ending amino acid information.

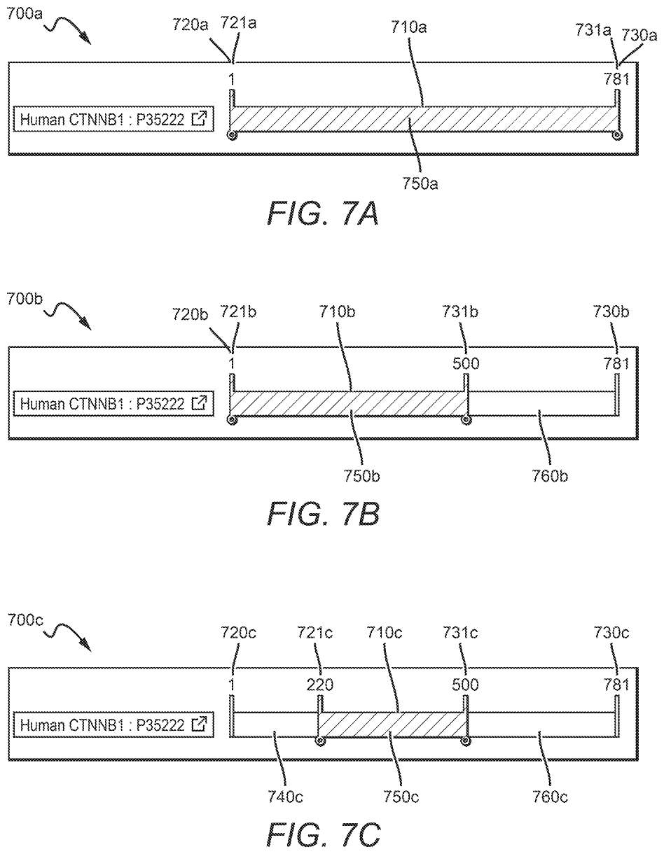

FIGS. 7A-7C are block diagrams illustrating interactive amino acid sequence sliders for generating a computer-implemented interactive graphical user interface for facilitating finding appropriate antibody products utilizing visual evaluation of immunogen or antigen information according to various embodiments. The examples illustrate filtering elements that are used to input the starting amino acid and ending amino acid for the immunogen or antigen of the target protein. In these examples, the target protein is human beta catenin and is 781 amino acids long. The slider elements are used to narrow the protein range from 781 to a desired length within a desired position or location relative to the target protein. The slider elements can move along the entire target protein range to identify the amino acid filtering values for the antibody product listing. When the sliders move, a shaded area representing the amino acid area of interest expands or contracts accordingly, to be within and in between the sliders, indicating a selected amino acid area of interest.

In some embodiments, any number of slider elements may be used to identify any number of intervals or ranges for amino acids of interest. In some embodiments, additional interface elements may be used to indicate filtering criteria or parameters with respect to amino acids of interest used to include or exclude antibody products from the antibody product listing, including for example, annotation interface elements, epitope indicator interface elements, phosphorylation site indicator interface elements or atomic structure or atomic coordinates interface elements used to indicate filtering criteria or parameters. In some embodiments, additional interface elements may indicate levels of protein structure including one of a primary protein structure, a secondary protein structure, a tertiary protein structure and a quaternary protein structure. In some embodiments, interface elements such as a dynamic picklist may be used within the computer-implemented interactive graphical user interface. The dynamic picklist may include for example, in a dropdown menu or set of buttons, one or more of a particular amino acid or an amino acid sequence range of the given target protein, and may be responsive to a user input to select at least one of the plurality of amino acid sequence ranges in the dropdown menu or set of radio buttons. Furthermore, in some embodiments, interface elements such as a numerical entry interface may be used within the computer-implemented graphical user interface. The numerical entry interface is responsive to a user input to manually enter, for example by typing in to a text entry box) a numerical position of a particular amino acid or a numerical range corresponding to an amino acid sequence range of the given target protein.

FIG. 7A is a block diagram illustrating interactive amino acid sequence slider interface component 700a, including interactive sequence slider 710a, N-terminus 720a, C-terminus 730a, left slider 721a, right slider 731a, selected area of interest 750a. Interactive component 700a is configured to operate or function as a filter for antibody products by filtering out antigens or immunogens based on an amino acid area of interest controlled or constrained by constraint indicators represented by sliders 721a and 731c.

In this example, sliders 721a and 731a are at amino acid positions 1 and 781, respectively. Slider 721a visually overlaps with N-terminus 720a and slider 731a visually overlaps with C-terminus 730a because the respective interface elements are at starting position of amino acid 1 and ending position of amino acid 781. Thus, only antibody products that were developed against amino acids within the target protein that are positioned between amino acid 1 and amino acid 781 (i.e., within selected area of interest 750a) will be filtered to be included in the product listing whereas any antibody products with immunogens or antigens derived from amino acids outside of that range will be filtered out. Although not shown, a deselected area may represent a range or interval of filtered out amino acids of interest.

FIG. 7B is a block diagram illustrating interactive amino acid sequence slider interface component 700b, including interactive sequence slider 710b, N-terminus 720b, C-terminus 730b, left slider 721b, right slider 731b, selected area of interest 750b, and deselected area 760b. Interactive component 700b is configured to operate or function as a filter for antibody products by filtering out antigens or immunogens based on an amino acid area of interest controlled by sliders 721b and 731c. In this example, sliders 721b and 731b are at amino acid positions 1 and 500, respectively. Slider 721b visually overlaps with N-terminus 720b because both interface elements are at starting position of amino acid 1. Thus, only antibody products that were developed against amino acids within the target protein that are positioned between amino acid 1 and amino acid 500 (i.e., within selected area of interest 750b) will be filtered to be included in the product listing whereas any antibody products with immunogens or antigens derived from amino acids outside of that range will be filtered out. The deselected area 760b represents the range or interval of filtered out amino acids of interest.

FIG. 7C is a block diagram illustrating interactive amino acid sequence slider interface component 700c, including interactive sequence slider 710c, N-terminus 720c, C-terminus 730c, left slider 721c, right slider 731c, selected area of interest 750c, deselected area 740c, and deselected area 760c. Interactive component 700c is configured to operate or function as a filter for antibody products by filtering out antigens or immunogens based on an amino acid area of interest controlled by sliders 721c and 731c. In this example, sliders 721c and 731c are at amino acid positions 220 and 500, respectively. Thus, only antibody products that were developed against amino acids within the target protein that are positioned between amino acid 220 and amino acid 500 (i.e., within selected area of interest 750c) will be filtered to be included in the product listing whereas any antibody products with immunogens or antigens derived from amino acids outside of that range will be filtered out. The deselected areas 740c and 760c represent the range or interval of filtered out amino acids of interest.

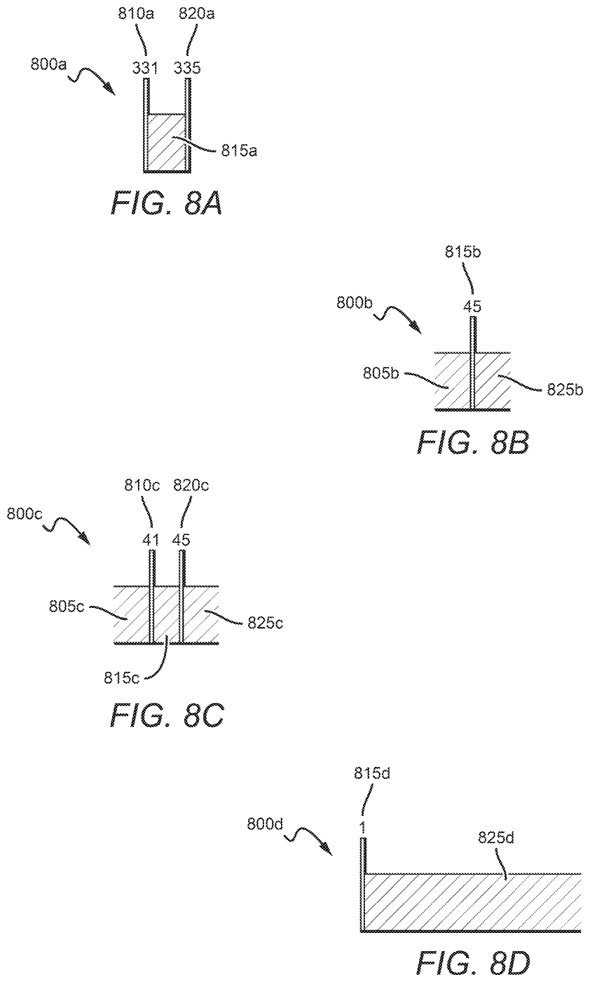

FIGS. 8A-8D are block diagrams illustrating antibody product infographics in a computer-implemented interactive graphical user interface for facilitating finding appropriate antibody products utilizing visual evaluation of immunogen or antigen information according to various embodiments.

FIG. 8A is a block diagram illustrating amino acid sequence infographic 800a, including starting amino acid 810a, ending amino acid 820a and area of interest 815a.

FIG. 8B is a block diagram illustrating amino acid sequence infographic 800b, including amino acid area 815b, amino acid area 805b and amino acid area 825b.

FIG. 8C is a block diagram illustrating amino acid sequence infographic 800c, including starting amino acid 810c, ending amino acid 820a, amino acid area of interest 815c, amino acid area 805c and amino acid area 825c.

FIG. 8D is a block diagram illustrating amino acid sequence infographic 800d, including starting amino acid 815d and amino acid area 825d.

FIGS. 8B-8D include additional estimated, undefined or ambiguous areas outside of the bounding or constraint elements. In some embodiments, these additional areas are visually indicated by fading out and away from the bounding elements. The additional undefined areas indicate a vicinity on the amino acid sequence without a defined start point or end point on the sequence. In such cases, the actual start and end points on the amino acid sequence are unknown, unavailable, or proprietary and which actual amino acids are included are not displayed. In some embodiments, the shaded area represents an estimated error tolerance range for included amino acids. In some embodiments, the shaded area represents a length of amino acids ranging from zero to twenty amino acids long.

For example, infographic 800d includes starting amino acid 815d that indicates the sequence starts at amino acid 1. Additional area 825d fades out from bounding element which is starting amino acid 815d. In some embodiments, the product description corresponding to this infographic 800d is accompanied by a text description for that antigen or immunogen that indicates that the antibody is derived from the N-terminus or an N terminal portion of the protein, in this case amino acid 1. That is, the antibody product starting point is known, but the end point is either not known or omitted, for example, as proprietary information of a third-party manufacturer or another entity. Also, as the antibody related to infographic 800d was made to an immunogen at the N-terminus, and which exact amino acids are included is not known or not provided by a third-party manufacturer, the end point is not defined and this is depicted with a fade out for infographic 800d.

FIG. 9 is a block diagram illustrating an interactive user interface for generating a computer-implemented interactive graphical user interface for facilitating finding appropriate antibody products utilizing visual evaluation of immunogen or antigen information according to various embodiments. In one embodiment, interactive interface 900 is a products results page for beta Catenin polyclonal antibody products.

The interactive interface 900 includes a search element 901, antigen link 902, navigation list 903, filter list 904, antibody product information 905, amino acid sequence filter 906, amino acid infographic 907, amino acid infographic 908, antigenic species filter 909, link 910, and immunogen information 911. Antigen link 902 is underlined, indicating that the antigen page is being displayed.

In this example, Link 910 includes the product web name which takes you to a product page. In this example, no top-row filter from filter list 904 is selected, and antigenic species filter 909 is defaulted to “Human.” In some embodiments, filters may include sliders, drop down menus or tags.

Amino acid infographic 907 illustrates an immunogen at N-terminus of beta Catenin. Amino acid infographic 908 illustrates an immunogen at amino acids 25-75 of beta Catenin. Filter 906 is illustrated as a full-length protein sequence providing filtering capability and a visual reference for comparing the results.

In some embodiments, the sort order for the product listing is based on at least one of sequence range information, product name, and price. In some embodiments, the sort based on sequence range information orders the products based on values for the leftmost bounding element, and the reverse sort would sort based on values for the rightmost bounding element. For example, in amino acid infographic 907, the leftmost bounding value is 1, as well as the two following products. In some embodiments, amino acid infographics may be expanded or zoomed into to provide more granular information regarding the amino acids of interest.

In some embodiments, the sort order for the product listing is based on the N-terminus or C-terminus. The default sort may be left to right, based on the lowest leftmost value. If the user does not filter or does not interact with the slider bar, then visually the infographics are visually aligned and sorted as moving from the left side of the protein to the right side.

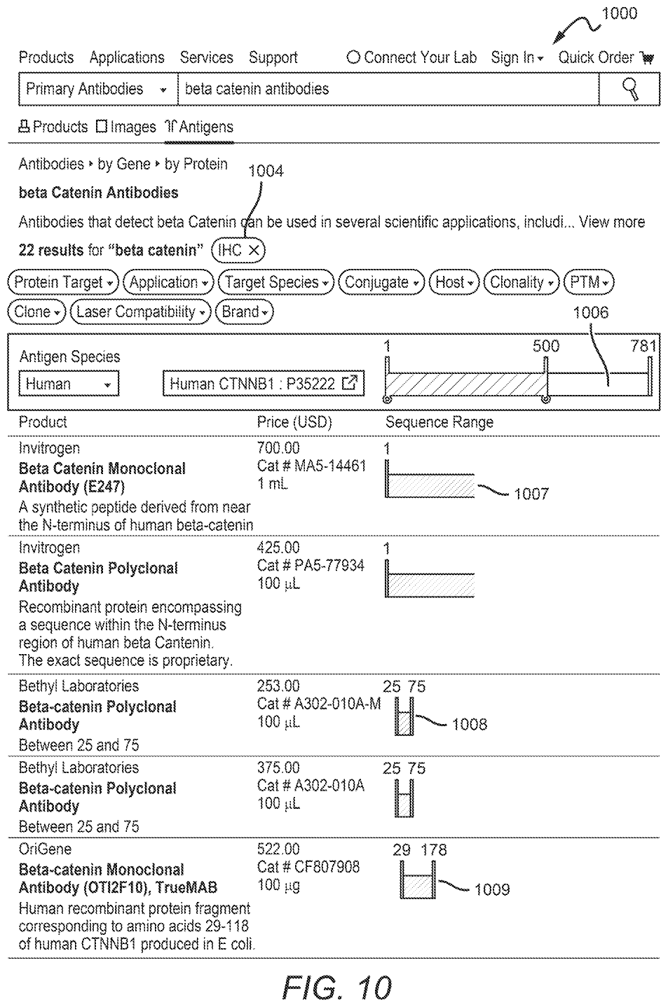

FIG. 10 is a block diagram illustrating an interactive user interface for generating a computer-implemented interactive graphical user interface for facilitating finding appropriate antibody products utilizing visual evaluation of immunogen or antigen information according to various embodiments. In one embodiment, interactive interface 1000 is a products results page for beta Catenin polyclonal antibody products. The interactive interface 1000 includes selection 1004, amino acid sequence filter 1006, amino acid infographics 1007-1009.

Amino acid infographic 1007 indicates that the antibody product was derived from or near the N-terminus of beta catenin. Amino acid infographic 1008 indicates that the antibody product was derived from between amino acids 25 and 75. Amino acid infographic 1009 indicates that the antibody product was derived from between amino acids 29 and 178.

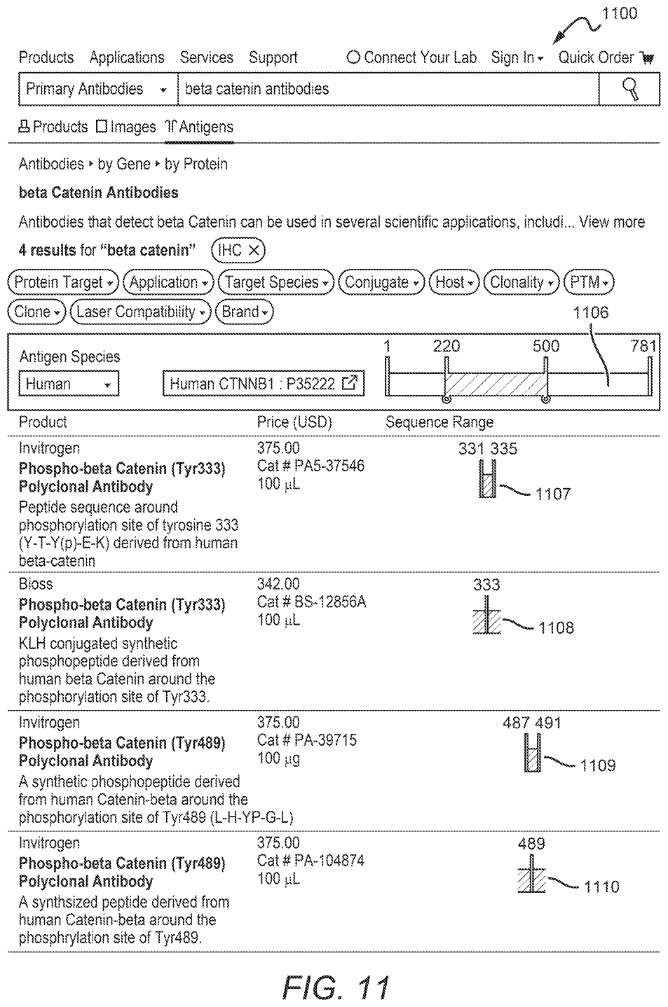

FIG. 11 is a block diagram illustrating an interactive user interface for generating a computer-implemented interactive graphical user interface for facilitating finding appropriate antibody products utilizing visual evaluation of immunogen or antigen information according to various embodiments. In one embodiment, interactive interface 1100 is a products results page for beta Catenin polyclonal antibody products. The interactive interface 1100 includes amino acid sequence filter 1106, amino acid infographics 1107-1110.

Amino acid infographic 1107 indicates that the antibody product was derived from amino acids between 331 and 335. Amino acid infographic 1108 indicates that the antibody product was derived around amino acid 333. Amino acid infographic 1109 indicates that the antibody product was derived from between amino acids 487 and 491. Amino acid infographic 1110 indicates that the antibody product was derived from around amino acid 489.

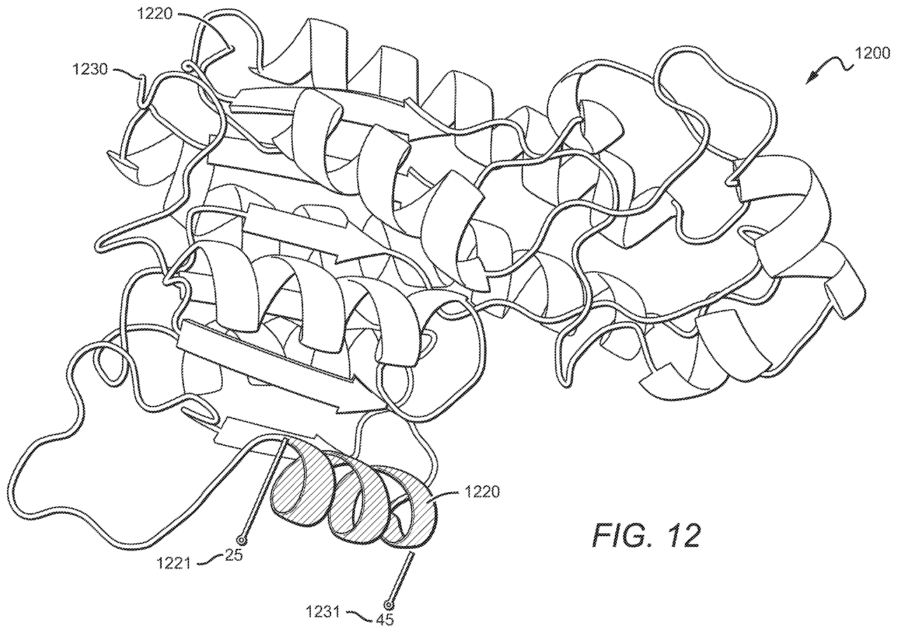

FIG. 12 is a block diagram illustrating a 3D interactive filter for facilitating finding appropriate antibody products utilizing visual evaluation of immunogen or antigen information according to various embodiments. In one embodiment, 3D interactive amino acid sequence slider interface component 1200 is visually displayed as a ribbon diagram, also known as a Richardson diagram, which is a 3D schematic representation of a protein structure. In this 3D display format, additional structural elements of the amino acids sequence can be visually indicated.

In this example, 3D interactive amino acid sequence slider interface component 1200 is illustrated as a ribbon that shows the overall path and organization of the protein in 3D and provides visual indication of details of the full atomic structure of the protein. The secondary structures of the full range amino acid sequence are illustrated within the ribbon, where the helices and beta sheets of a protein are shown by corkscrews and arrows respectively. In some embodiments, alpha helices are shown as coiled ribbons or thick tubes, and beta strands as arrows, and non-repetitive coils or loops as lines or thin tubes. The direction indication of the amino acid chain is indicated by the arrows. In some embodiments, the direction may be indicated overall by a color ramp, color gradient, pattern density, or other visual indication along the length of the ribbon. Further, 3D interactive amino acid sequence slider interface component 1200 provides visual indication for whether the amino acid area of interest comprises coils, turns, strands, helices, and other visual indications of molecular structure, including twists, folds and unfolds.

In this example, 3D interactive amino acid sequence slider interface component 1200 includes N-terminus 1220, C-terminus 1230, left slider 1221, right slider 1231, and selected area of interest 1202. In this example, the filter slider corresponds to the filter slider illustrated in FIG. 7C. FIG. 7C is illustrated as a straight line of a full range sequence of amino acids whereas FIG. 12 is illustrated as a 3D schematic representation of a full range sequence of amino acids.

Slider elements 1221 and 1220 can move around all along the protein ribbons to identify the filtering values for the product listing. When the sliders move, the shaded area expands or contracts accordingly, to be within and in between the sliders, indicating a selected amino acid area of interest.

In some embodiments, the amino acid values are dynamically updated when the slider values are moved. In this example, the amino acid value for slider 1221 is 25 and the amino acid value for slider 1231 is 45. When slider 1221 is moved along the ribbon generally toward the N-terminus 1220, for example, following the direction indications of the protein described above, the value decreases until it reaches 1 at 1220. In some embodiments, the direction indication may be determined by moving sliders and observing the amino acid number values decrease or increase. When slider 1231 is moved along the ribbon generally toward the C-terminus 1230, the amino acid value increases from 45. In some embodiments, the 3D interactive filter is represented in full 3D using virtual reality or augmented reality technology.

In some embodiments, 3D interactive amino acid sequence slider interface component 1200 is rotatable in response to a user event, including for example a click and/or drag on the structure to turn the interface component 1200. In some embodiments, interface component 1200 is represented in full 3D using virtual reality or augmented reality technology. In some embodiments, a user may, for example, tap, pinch out, and/or click on the structure to zoom in more closely on 3D interactive amino acid sequence slider interface component 1200 and/or make interface component 1200 larger. In some embodiments, a user may zoom out on interface component 1200 by, for example, pinching, tapping, and/or clicking on the structure to make interface component 1200 smaller and/or fit into the screen. In some embodiments, a user may manually enter or choose from a picklist or dropdown menu of percentages a higher percentage of an “actual” or “full” size to zoom in more closely on interface component 1200, or manually enter or choose from a picklist or a dropdown menu of percentages, a lower percentage of an “actual” or “full” size to zoom out and make interface component 1200 smaller and/or fit onto the screen.

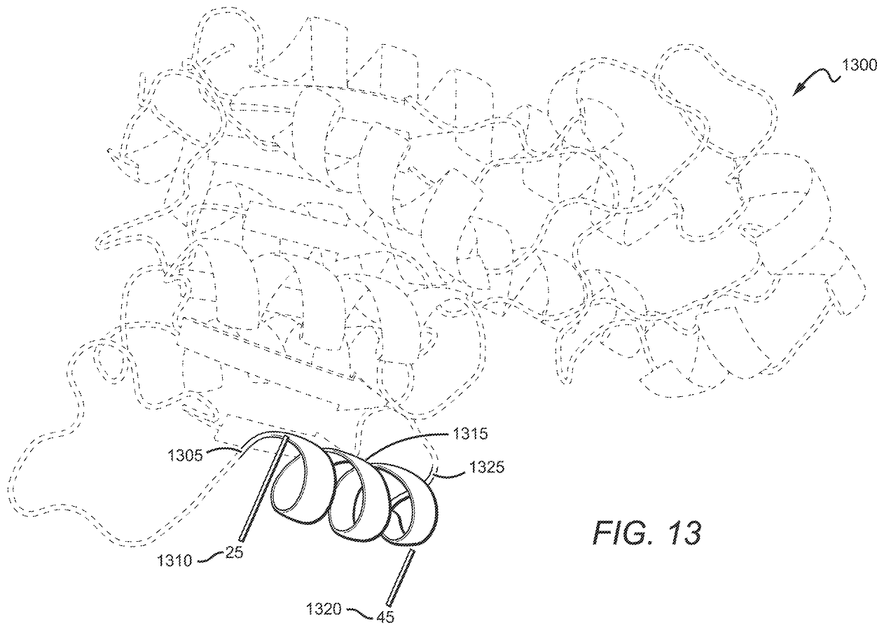

FIG. 13 is a block diagram illustrating a 3D infographic related to an antibody product listing in an interactive 3D graphical user interface for facilitating finding appropriate antibody products utilizing visual evaluation of immunogen or antigen information according to various embodiments. In one embodiment, infographic 1300 facilitates finding appropriate antibody products utilizing visual evaluation of immunogen or antigen information. In this example, the 3D infographic corresponds to the amino acid sequence infographic illustrated in FIG. 8C in that they both illustrate infographics including estimated amino acid values.

In one embodiment, 3D amino acid sequence infographic 1300 is visually displayed as a ribbon diagram, also known as a Richardson diagram, which is a 3D schematic representation of a protein structure. In this 3D display format, additional structural elements of the amino acids sequence can be visually indicated. In this example, infographic 1300 is illustrated as a ribbon that shows the overall path and organization of the protein in 3D and provides visual indication for details of the full atomic structure of the protein. The secondary structures of the full range amino acid sequence are illustrated within the ribbon, where the helices and beta sheets of a protein are shown by corkscrews and arrows respectively. In some embodiments, alpha helices are shown as coiled ribbons or thick tubes, and beta strands as arrows, and non-repetitive coils or loops as lines or thin tubes. The direction indication of the amino acid chain is indicated by the arrows. In some embodiments, the direction may be indicated overall by a color ramp, color gradient, pattern density, or other visual indication along the length of the ribbon. Further, infographic 1300 provides visual indication for whether the amino acid area of interest comprises coils, turns, strands, helices, and other visual indications of molecular structure, including twists, folds and unfolds.

In one embodiment, 3D amino acid sequence infographic 1300 includes amino acid estimated area 1305, amino acid left boundary 1310, amino acid area of interest 1315, amino acid right boundary 1320, and amino acid estimated area 1325. Amino acid left boundary 1310 and amino acid right boundary 1320 indicate the bounding limits for amino acid area of interest 1315. In some embodiments, boundary elements may be estimated values and not concrete or limiting boundaries.

Infographic 1300 is a 3D infographic that includes the entire structure of a protein and a specific amino acid range within that protein spanning residues 25-45. The amino acid range may be selected and/or identified by movable lines pointing to each residue location in the protein structure. Infographic 1300 is illustrated in a monochromatic color scheme throughout the protein structure. In some embodiments, a color scheme may be used. Infographic 1300 includes a visually distinguished immunogen range 25-45. In some embodiments, visual distinguishing may be accomplished using a different color, a different pattern, or a boundary highlight. Infographic 1300 includes all of the features for amino acid sequence infographics described herein.

In some embodiments, infographic 1300 is rotatable in response to a user event, including for example a click and/or drag on the structure to turn the infographic. In some embodiments, infographic 1300 is represented in full 3D using virtual reality or augmented reality technology. In some embodiments, a user may, for example, tap, pinch out, and/or click on the structure to zoom in more closely on 3D amino acid sequence infographic 1300 and/or make infographic 1300 larger. In some embodiments, a user may zoom out on infographic 1300 by, for example, pinching, tapping, and/or clicking on the structure to make infographic 1300 smaller and/or fit into the screen. In some embodiments, a user may manually enter or choose from a picklist or dropdown menu of percentages a higher percentage of an “actual” or “full” size to zoom in more closely on infographic 1300, or manually enter or choose from a picklist or a dropdown menu of percentages, a lower percentage of an “actual” or “full” size to zoom out and make infographic 1300 smaller and/or fit onto the screen.

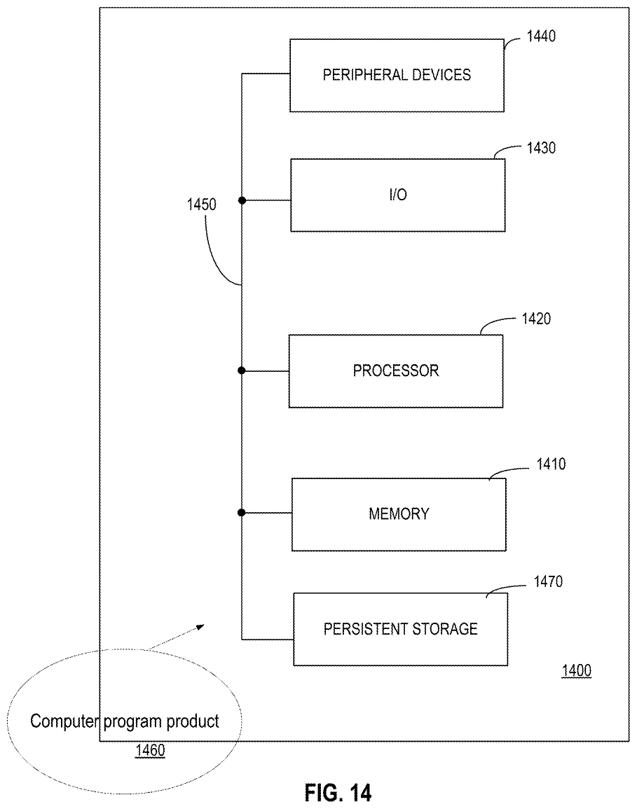

FIG. 14 illustrates an exemplary computer system configurable by a computer program product to carry out embodiments of the present invention.

In the example, computer system 1400 may provide one or more of the components of an interactive user interface for generating a computer-implemented interactive graphical user interface for facilitating finding appropriate antibody products utilizing visual evaluation of immunogen or antigen information. Computer system 1400 executes instruction code contained in a computer program product 1460 (which may, for example, be part of the interactive 3D graphical user interface for facilitating finding appropriate antibody products utilizing visual evaluation of immunogen or antigen information as discussed herein). Computer program product 1460 comprises executable code in an electronically readable medium that may instruct one or more computers such as computer system 1400 to perform processing that accomplishes the exemplary method steps performed by the embodiments referenced herein. The electronically readable medium may be any non-transitory medium that stores information electronically and may be accessed locally or remotely, for example, via a network connection. In alternative embodiments, the medium may be transitory. The medium may include a plurality of geographically dispersed media, each configured to store different parts of the executable code at different locations or at different times. The executable instruction code in an electronically readable medium directs the illustrated computer system 1400 to carry out various exemplary tasks described herein. The executable code for directing the carrying out of tasks described herein would be typically realized in software. However, it will be appreciated by those skilled in the art that computers or other electronic devices might utilize code realized in hardware to perform many or all the identified tasks without departing from the present invention. Those skilled in the art will understand that many variations on executable code may be found that implement exemplary methods within the spirit and the scope of the present invention.

The code or a copy of the code contained in computer program product 1460 may reside in one or more storage persistent media (not separately shown) communicatively coupled to computer system 1400 for loading and storage in persistent storage device 1470 and/or memory 1410 for execution by processor 1420. Computer system 1400 also includes I/O subsystem 1430 and peripheral devices 1440. I/O subsystem 1430, peripheral devices 1440, processor 1420, memory 1410, and persistent storage device 1470 are coupled via bus 1450. Like persistent storage device 1470 and any other persistent storage that might contain computer program product 1460, memory 1410 is a non-transitory media (even if implemented as a typical volatile computer memory device). Moreover, those skilled in the art will appreciate that in addition to storing computer program product 1460 for carrying out the processing described herein, memory 1410 and/or persistent storage device 1470 may be configured to store the various data elements referenced and illustrated herein.

Those skilled in the art will appreciate computer system 1400 illustrates just one example of a system in which a computer program product in accordance with an embodiment of the present invention may be implemented. To cite but one example of an alternative embodiment, storage and execution of instructions contained in a computer program product in accordance with an embodiment of the present invention may be distributed over multiple computers, such as, for example, over the computers of a distributed computing network.

Although some of the various embodiments presented herein constitute a single combination of inventive elements, it should be appreciated that the inventive subject matter is considered to include all possible combinations of the disclosed elements. As such, if one embodiment comprises elements A, B, and C, and another embodiment comprises elements B and D, then the inventive subject matter is also considered to include other remaining combinations of A, B, C, or D, even if not explicitly discussed herein. Further, the transitional term “comprising” means to have as parts or members, or to be those parts or members. As used herein, the transitional term “comprising” is inclusive or open-ended and does not exclude additional, unrecited elements or method steps.

Any process described herein may be performed in any order and may omit any of the steps in the process. Processes may also be combined with other processes or steps of other processes. Although steps or operations may be described as a sequential process, some of the steps or operations may in fact be performed in parallel, concurrently, and/or in a distributed environment, and with program code stored locally or remotely for access by single or multi-processor machines. In addition, in some embodiments the order of steps or operations may be rearranged without departing from the spirit of the disclosed subject matter.

Throughout the discussion herein, numerous references are made regarding clouds, servers, services, devices, platforms, frameworks, cyber physical systems, or other systems formed from computing devices. It should be appreciated that the use of such terms is deemed to represent at least one or more computing devices having at least one processor, for instance but not limited to an application-specific integrated circuit (ASIC), field-programmable gate array (FPGA), digital signal processor (DSP), x86, reduced instruction set computer architecture (ARM), ColdFire, graphics processing unit (GPU), and a multi-core processors configured to execute software instructions stored on a computer readable tangible, non-transitory medium for instance but not limited to a hard drive, solid state drive, random-access memory (RAM), flash, and read only memory (ROM) among other components. For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable medium storing the instructions that cause a processor to execute the disclosed steps. The various servers, systems, databases, or interfaces can exchange data using standardized protocols or algorithms, possibly based on Hypertext Transfer Protocol (HTTP), secure Hypertext Transfer Protocol (HTTPS), Advanced Encryption Standard (AES), public-private key exchanges, web service Application programming interfaces (APIs), known financial transaction protocols, or other electronic information exchanging methods. Data exchanges can be conducted over a packet-switched network, a circuit-switched network, the Internet, Local area network (LAN), wide area network (WAN), virtual private network (VPN), or other type of network.

A system, server, device, model, or other computing element according to some embodiments, being configured to perform or execute functions on data in a memory, where the meaning of “configured to” or “programmed to” is defined as one or more processors or cores of the computing element being programmed by a set of software instructions stored in the memory of the computing element to execute the set of functions on target data or data objects stored in the memory.

It should be noted that any language directed to a computing device should be read to include any suitable combination of computing devices, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium for instance but not limited to a hard drive, field programmable gate array (FPGA), programmable logic array (PLA), solid state drive, RAM, flash, and ROMThe software instructions configure or program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Systems, devices, and methods described herein may be implemented using a computer program product tangibly embodied in an information carrier, for example in a non-transitory machine-readable storage device, for execution by a programmable processor; and the method steps described herein, including for example one or more of the steps of FIGS. 1 and 2 may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Although the computing devices described herein such as but not limited to network nodes, cloud-based models, and virtual machines may include the illustrated combination of hardware components, other embodiments may comprise computing devices with different combinations of components. It is to be understood that these computing devices may comprise any suitable combination of hardware and/or software needed to perform the tasks, features, functions and methods disclosed herein. Determining, calculating, obtaining or similar operations described herein may be performed by processing circuitry, which may process information by, for example, converting the obtained information into other information, comparing the obtained information or converted information to information stored in the network node, and/or performing one or more operations based on the obtained information or converted information, and as a result of said processing making a determination. Moreover, while components are depicted as single boxes located within a larger box, or nested within multiple boxes, in practice, computing devices may comprise multiple different physical components that make up a single illustrated component, and functionality may be partitioned between separate components. For example, a communication interface may be configured to include any of the components described herein, and/or the functionality of the components may be partitioned between the processing circuitry and the communication interface. In another example, non-computationally intensive functions of any of such components may be implemented in software or firmware and computationally intensive functions may be implemented in hardware.

In certain embodiments, some or all of the functionality described herein may be provided by processing circuitry executing instructions stored on in memory, which in certain embodiments may be a computer program product in the form of a non-transitory computer-readable storage medium. In alternative embodiments, some or all of the functionality may be provided by the processing circuitry without executing instructions stored on a separate or discrete device-readable storage medium, such as in a hard-wired manner. In any of those particular embodiments, whether executing instructions stored on a non-transitory computer-readable storage medium or not, the processing circuitry can be configured to perform the described functionality. The benefits provided by such functionality are not limited to the processing circuitry alone or to other components of the computing device but are enjoyed by the computing device as a whole, and/or by end users and a wireless network generally.

The disclosed technology is designed to be compatible with and operable by any computing device, including, for example, a desktop computer, a mobile device, a smart phone, an Internet of Things device, an Augmented Realty or Virtual Reality device, personal digital assistant (PDA), gaming console or device, playback appliance, wearable terminal device, mobile station, tablet, laptop, or a combination thereof.

While some examples described herein may refer to functions performed by given actors such as “users,” “systems,” and/or other entities, it should be understood that this is for purposes of explanation only. The claims should not be interpreted to require action by any such example actor unless explicitly required by the language of the claims themselves.

Many of the details, dimensions, angles and other features shown in the Figures are merely illustrative of particular embodiments of the disclosed technology. Accordingly, other embodiments can have other details, dimensions, angles and features without departing from the spirit or scope of the disclosure. In addition, those of ordinary skill in the art will appreciate that further embodiments of the various disclosed technologies can be practiced without several of the details described below.

Throughout the specification and claims, the following terms take the meanings explicitly associated herein, unless the context clearly dictates otherwise:

The phrase “in one embodiment” as used herein does not necessarily refer to the same embodiment, though it may. Thus, as described below, various embodiments of the invention may be readily combined, without departing from the scope or spirit of the invention.

As used herein, the term “or” is an inclusive “or” operator and is equivalent to the term “and/or,” unless the context clearly dictates otherwise.

The term “based on” is not exclusive and allows for being based on additional factors not described, unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of a networked environment where two or more components or devices are able to exchange data, the terms “coupled to” and “coupled with” are also used to mean “communicatively coupled with”, possibly via one or more intermediary devices.

In addition, throughout the specification, the meaning of “a”, “an”, and “the” includes plural references, and the meaning of “in” includes “in” and “on”.

While certain illustrative embodiments are described herein, those embodiments are presented by way of example only, and not limitation. While the embodiments have been particularly shown and described, it will be understood that various changes in form and detail may be made. Although various embodiments have been described as having features and/or combinations of components, other embodiments are possible having a combination of any features and/or components from any of embodiments as discussed above.

1. A method for generating a computer-implemented interactive graphical user interface for facilitating finding appropriate antibody products utilizing visual evaluation of sequence information, the method comprising:
generating an antibody product listing within the graphical user interface, responsive to a user interaction, wherein the antibody product listing includes at least one antibody product including corresponding sequence information and product specifications;
generating a sequence infographic based on the corresponding sequence information, wherein the sequence infographic includes an amino acid sequence area of interest indicator; and
displaying the sequence infographic aligned relative to a plurality of sequence infographics.

2. The method of claim 1, wherein the amino acid sequence area of interest indicator includes an epitope indicator shown relative to the sequence amino acid sequence area of interest indicator.

3. The method of claim 2, wherein the sequence infographic includes a base layer representing an amino acid sequence range of a given target protein, a second layer representing the amino acid sequence area of interest indicator that includes visual elements related to data points in the amino acid sequence range of the given target protein, and a third layer representing an epitope.

4. (canceled)

5. The method of claim 3, wherein the visual elements include at least one of a first visual indicator representing a specific data point, a second visual indicator representing an interval between two data points, a third visual indicator representing an estimated data point, a fourth visual indicator representing an estimated interval including at least one estimated data point.

6. The method of claim 1, wherein the amino acid sequence area of interest indicator represents at least one of a particular amino acid sequence value, an amino acid sequence range of values, an amino acid sequence starting value, an amino acid sequence ending value, and an amino acid sequence intermediate value.

7. The method of claim 1, further comprising:
detecting an input indicating a movement of a bounding element related to the amino acid sequence area of interest, wherein the bounding element represents an amino acid bounding value; and
updating the antibody product listing within the graphical user interface responsive to the input by excluding antibody products with at least one amino acid sequence range value outside the amino acid bounding value,
wherein the bounding element includes a slider visually positioned on an interval representing values of the amino acid sequence range.

8-9. (canceled)

10. The method of claim 7, wherein location of the slider on the interval indicates a bounding value in the amino acid sequence range of antibody products included in the antibody product listings.

11. The method of claim 7, wherein moving the slider from a first location to a second location indicates a change from a first bounding value to a second bounding value.

12. (canceled)

13. The method of claim 1, wherein displaying further comprises aligning each sequence infographic of the plurality of sequence infographics along the visual representation of the amino acid sequence range of the given target protein.

14. The method of claim 1, wherein the sequence infographic enables visual evaluation of the sequence based on at least one of sequence size, sequence position, and sequence overlap.

15. (canceled)

16. The method of claim 1, wherein generating the sequence infographic further comprises generating a three-dimensional model of the sequence infographic including the amino acid sequence area of interest indicator.

17. The method of claim 1, further comprising:
generating a multi-dimensional protein model representing the plurality of sequence infographics; and
visually distinguishing each sequence infographic of the plurality of sequence infographics as a layer of the multi-dimensional model.

18. (canceled)

19. The method of claim 17, further comprising visually aligning each layer along the multi-dimensional model relative to a corresponding amino acid sequence for each layer.

20. The method of claim 1, further comprising generating a multi-dimensional protein model including a visual representation of at least one level of protein structure, wherein the at least one level of protein structure includes one of a primary protein structure, a secondary protein structure, a tertiary protein structure and a quaternary protein structure.

21. (canceled)

22. The method of claim 20, further comprising positioning the amino acid sequence area of interest indicator on the at least one level of protein structure.

23-52. (canceled)

53. A method for generating a computer-implemented interactive graphical user interface for facilitating finding appropriate antibody products utilizing visual evaluation of sequence information, the method comprising, within the computer-implemented graphical user interface:
generating a dynamic amino acid sequence filter element including at least one movable bounding indicator, wherein the dynamic filter element visually represents an amino acid sequence range of a given target protein, and wherein the at least one movable bounding indicator visually represents a particular amino acid within the amino acid sequence range; and
visually distinguishing an amino acid sequence area of interest within the dynamic amino acid sequence filter element based on a position of the at least one movable bounding indicator on the dynamic amino acid sequence filter element corresponding to the particular amino acid.

54. The method of claim 53, further comprising updating an antibody product listing within the graphical user interface responsive to a user input by excluding antibody products with at least one amino acid sequence range value outside the amino acid sequence area of interest.

55. The method of claim 53, wherein the at least one movable bounding indicator includes a slider visually positioned on an edge of the amino acid sequence area of interest, and wherein moving the slider from a first location to a second location indicates a change to a length of the amino acid sequence area of interest.

56. The method of claim 53, further comprising a dynamic picklist within the computer-implemented interactive graphical user interface, wherein the dynamic picklist includes one or more of a particular amino acid or an amino acid sequence range of the given target protein, and is responsive to a user input to select at least one of the particular amino acid or amino acid sequence range.

57. The method of claim 53, further comprising a numerical entry interface within the computer-implemented graphical user interface, wherein the numerical entry interface is responsive to a user input to manually enter one or more of a particular amino acid or an amino acid sequence range of the given target protein.
